(19) United States
(12) Patent Application Publication
(10) Pub. No.: US 2015/0174279 A1
(43) Pub. Date: Jun. 25, 2015

(54) TOPICAL DRESSING TO FACILITATE WOUND RECOVERY

(71) Applicant: Fayetteville State University, Fayetteville, NC (US)

(72) Inventor: Carla Raineri Padilla, Fayetteville, NC (US)

(73) Assignee: FAYETTEVILLE STATE UNIVERSITY, Fayetteville, NC (US)

(21) Appl. No.: 14/538,229

(22) Filed: Nov. 11, 2014

Related U.S. Application Data
(60) Provisional application No. 61/918,037, filed on Dec. 19, 2013.

(51) Int. Cl. A61L 15/18; A61L 15/44; A61L 15/40; A61L 15/52; A61L 15/22

(52) U.S. Cl. CPC A61L 15/18; A61L 15/52; A61L 15/22; A61L 15/40; A61L 15/44

(57) ABSTRACT

A topical dressing for wound recovery is disclosed. The dressing includes a water impermeable air side layer, a water permeable wound-side layer, and a gel based upon a silicon oxide composition positioned between the air side layer and the wound side layer.

### BACKGROUND

The present invention relates to the treatment of wounds with a dressing and in a manner that enhances recovery.

Wounds fall into a variety of categories which include, but are not limited to penetrating wounds (both accidental and intentional; e.g., surgical), non-penetrating wounds, thermal wounds (burns), chemical and electrical wounds, wounds from acute causes such as trauma, and wounds from chronic causes such as disease or chronic pressure (pressure ulcers or bed sores).

From a diagnosis and treatment standpoint, many wounds (and their effective treatment) are similar in both humans and animals. Depending upon the severity of the wound, the problems or consequences can be as minimal as temporary discomfort, but can escalate to permanent injury or loss of life.

When medical treatment is immediately or quickly available, wound care typically follows a known, helpful regimen. As an example, the wound and patient are initially assessed, and the wound is characterized by type, size and other characteristics. The wound may be cleaned at this point. Next, the available medical personnel make a determination of the needed treatment and the potential for infection. The wound may be cleaned at this point as well, and topical anesthetics, antibiotics, analgesics or other medications may be applied. In some cases, the medications are applied directly to the wound especially in the case of common anti-microbial agents (bacitracin) silver-based medications and sulfur-based medications (among others). As a typical last step, a dressing or bandage is applied.

For a number of reasons, including the type(s) of wound and its location and extent, the recovery from patient to patient can be highly variable. In many cases wound treatment may need to be carried out over a relatively long recovery phase. As a result, it remains necessary throughout recovery to protect the wound, to prevent or treat infection, and to promote good healing (e.g., to avoid or minimize scarring and other undesired outcomes).

Accordingly, wound treatment presents challenges such as preventing (or treating) infection, avoiding further physical damage, minimizing pain, and (depending upon the wound size, type and location) moderating or preventing loss of water or electrolytes. As obstacles to these efforts, if dressings and bandages are dry when applied, they can have a tendency to adhere or otherwise stick to wounds. In some cases, the act of changing the dressing on an open wound can cause significant loss of body fluids, with accompanying loss of electrolytes. Furthermore, the ongoing need for repeated dressing changes leads to a higher potential for water and electrolyte loss as well as infection. This places additional time and activity stress on the medical staff, particularly the nursing staff.

In turn these factors can cause pain and additional damage to a wound whenever the bandage or dressing is removed or changed. Furthermore, topical delivery of medications to the wound site can be challenging while a wound is healing, and dressed in particular.

### SUMMARY

In one aspect the invention is a topical dressing for wound recovery. The dressing includes a water impermeable air side layer, a water permeable wound-side layer, and a gel based upon a silicon oxide composition (most frequently silicon dioxide) positioned between the air side layer and the wound side layer.

In another aspect, the invention is a topical dressing for wound recovery that includes an air side water impermeable layer, a water permeable wound side layer, an adhesive layer on the wound side face of the air side layer, and a desiccated silicon oxide gel precursor on the adhesive layer.

In yet another aspect the invention is a method of treating a wound that includes the steps of adding water to a desiccated silicon oxide-based gel precursor based upon a silicon oxide composition maintained in a dressing between an air side water impermeable layer and a water permeable wound side layer, and applying the wound side layer of the dressing to a wound.

In yet another aspect the invention is a method of treating a wound that includes the steps of applying a dressing to a wound, and in which the dressing contains a hydrated gel that has been hydrated by adding water to a desiccated gel precursor based upon a silicon oxide composition in which the desiccated gel precursor is maintained between an air side water impermeable layer and a water permeable wound side layer in a wound dressing.

The foregoing and other objects and advantages of the invention and the manner in which the same are accomplished will become clearer based on the followed detailed description taken in conjunction with the accompanying drawings.

### BRIEF DESCRIPTION OF THE DRAWINGS

FIG. 1 is a perspective view of the form of a patient in relation to the dressing of the invention.

FIG. 2 is an exploded view of the dressing of the invention.

FIG. 3 is a cross-sectional view of one embodiment of the invention.

FIG. 4 is a cross-sectional view of a second embodiment of the invention

### DETAILED DESCRIPTION

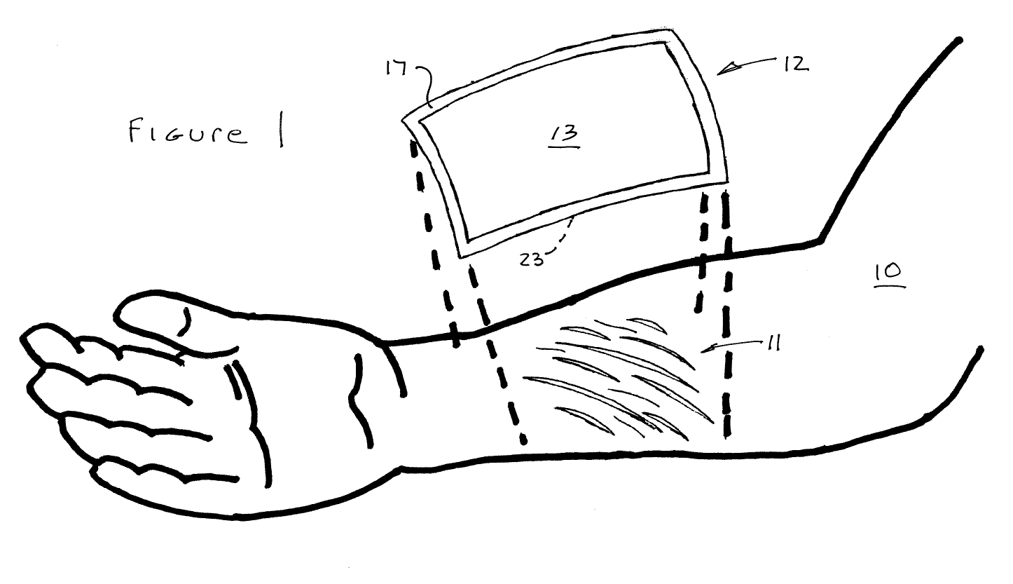

FIG. 1 illustrates a topical dressing broadly designated at 12 for wound recovery in the context of an arm 10 and a wound 11 on the arm 10. As set forth with respect to the other drawings, the topical dressing 12 is formed of several layers, two or more of which can be joined together by a seal 17 which can be accomplished using heat (e.g., for thermosetting or thermoplastic polymers); or with an adhesive that is otherwise compatible with the materials and the medical application; or mechanically such as with fasteners or by forcing the layers together with an instrument that joins them In addition to structural integrity, the seal 17 helps prevent gel loss or undesired drying of the gel in use.

For the sake of clarity, the seal 17 is not specifically illustrated in FIGS. 2-4, but is present in physical embodiments.

FIG. 2 is an exploded view of the three primary layers that make up the topical dressing 12. The first layer is a water impermeable air side layer 13 and the second layer is a water permeable wound side layer 15. A noncytotoxic gel 14 based on a silicon oxide composition is positioned between the air side layer 13 and the wound side layer 15. It will be understood that FIGS. 2-4 are schematic in nature and not necessarily to scale, but in most cases the area of the dressing will be proportionally much greater than its thickness.

An exemplary silicon oxide composition is fumed amorphous silica. Fumed silica is pure silicon dioxide (SiO₂) typically formed by oxidizing vaporized silicon tetrachloride in a high temperature flame with excess hydrogen and oxygen. Fumed silica is also known as pyrogenic silica and is characterized as non-crystalline, with very fine grain and much less density (and much more surface area) than ordinary silicon dioxide.

Fumed silica can also be produced by vaporizing quartz sand at high temperatures (e.g. 3000° C.).

In typical commercial use, fumed silica can act as a universal thickening agent, a thickening agent in food products, and an anti-caking agent in powdered products. It has similar desiccant properties to silica gel and in some circumstances can be used as a light abrasive (e.g., toothpaste).

Fumed silica suitable for purposes of the invention is available under the name “AEROSIL” from Evonik Degussa Corporation, Parsippany, N.J. 07054 USA. It is categorized under Chemical Abstracts Service Registry No. 67256-35-3. Some types of fumed silica are also known as “cab-o-sil,” or “cabosil,” “synthetic amorphous silica” or “pyrogenic amorphous silica.”

Fumed silica has a very fine particle size (typically 5-50 nanometers) and is categorized as an aerogel. It contains about 94% dead air space and has a density of about 2.3 pounds per cubic foot (160-190 kg/m³). The particles typically have a surface area of between about 50 and 600 m²/g.

The term “gel” is used here in a somewhat broad sense. For example, Lewis, HAWLEYS CONDENSED CHEMICAL DICTIONARY, 11th Edition, von Nostrand Reinhold (1987) defines a gel as a colloid in which the dispersed phase has combined with the continuous phase to produce a viscous jelly-like product. According to this definition, “a gel is made by cooling a solution whereupon certain kinds of solutes (gelatin) form sub microscopic crystalline particle groups which retain much solvent in the interstices.”

The present invention is not limited to this dictionary definition of “gel.”

Without being bound by theory, it appears that the fumed silica is absorbing water in a physical relationship to form a semi-solid composition that has many of the properties of a classically-defined gel, but which does not necessarily include gelatin (proteins) and does not necessarily form sub microscopic crystalline particle groups in the manner set forth in the formal definition.

Other conventional gel forming materials such as the natural products (e.g., agar and related products and derivatives) will form an initial usable gel. Once the gel is formed, however, it generally is not possible to dehydrate the gel and reconstitute the gel-forming material. Thus, for example, agar, once it has been gelled, is sufficiently changed from its starting composition that it cannot be successfully reused even if another nutrient solution was added to it.

Stated differently, once an agar gel is formed and used the original agar cannot be recovered or reused.

The viscosity of the gel can be controlled by controlling the ratio of silica to liquid, preferably within the ranges set forth herein.

The pH of gels formed from fumed amorphous silica tends to be acidic (often 3.0-4.0). Thus, the gel can be buffered as necessary or desired to keep the pH at or near neutral (pH=7) or another pH. Buffering from 3 to 8 is typical depending upon the circumstances. Normal human blood has a pH of 7.2 and thus buffering is often used to reach that point. In other circumstances a different pH can be advantageous (e.g., a pH below 7—more acidic—helps fight certain types of bacteria). The gel can be buffered as desired using compositions that otherwise do not interfere with the structure or operation of the dressing or generate any undesired interaction with a typical wound. Appropriate buffers include sodium bicarbonate, calcium carbonate, magnesium oxide, monosodium phosphate, bisodium phosphate, or other simple salts of moderate acids or bases and their conjugates

The air side layer 13 can be selected from the group consisting of polymers, metal foils, combinations of polymers, and combinations of polymers and metal foils. For example, thin layers of metals deposited on polyester are well understood to provide appropriate barrier properties for many purposes.

In particular the air side layer is frequently a polymer, appropriate examples of which include polyester, fluorinated ethylene polymers (PTFE, Teflon®, etc.), various grades of polyethylene and polypropylene, polyamides, ethylene vinyl alcohol, and combinations of these.

Aluminum foil is generally impermeable to gas and water vapor (moisture) above a thickness of about 20 μm. It has the advantage of desirable “dead fold” characteristics (it maintains its shape when wrapped or placed on an object).

More typically in medical applications, a thin layer of foil is combined with one or more polymers to form the desired impermeable barrier. Examples include a layer of foil sandwiched between polyester (PET) and polyethylene (PE), or between polypropylene (PP) and polyethylene or between polyester and polypropylene. The nature, variations, and uses of these polymers—e.g., high density polyethylene (HDPE), low density polyethylene (LDPE), biaxially oriented polypropylene (BOPP), and a number of others—are well understood in the art and will not be discussed in detail here.

Other impermeable coatings can be formed without aluminum or another metal foil. BOPP has a favorable low density (0.9 g/m³) a relatively high melting point (169° C., which permits sterilization) and forms an excellent moisture barrier.

Polyester has outstanding tear resistance, hard transparency and excellent printability when a labeled item is important.

Polyamides (nylons) have high melting points (e.g., 220° C.), high impact resistance, and high tensile strength. The moisture transmission rate is relatively high, however, and increases with the moisture content of the material. Therefore, the polyamides are typically used in combination with a second barrier material (e.g. polyethylene or polypropylene).

Ethylene vinyl alcohol resins (EVOH) have excellent gas barrier and chemical resistance properties, but these barrier properties deteriorate quickly in humid conditions. As a result, EVOH films are usually embedded in polyolefin layers to obtain the necessary moisture barrier.

Low density polyethylene and its derivatives have excellent water barrier properties, and good chemical resistance to acids and alkalis, but are more sensitive to hydrocarbons, oils and greases. As is recognized by the art, the exact properties of low density polyethylene (LDPE) depend to a significant extent on the manner in which the polymerization is carried out.

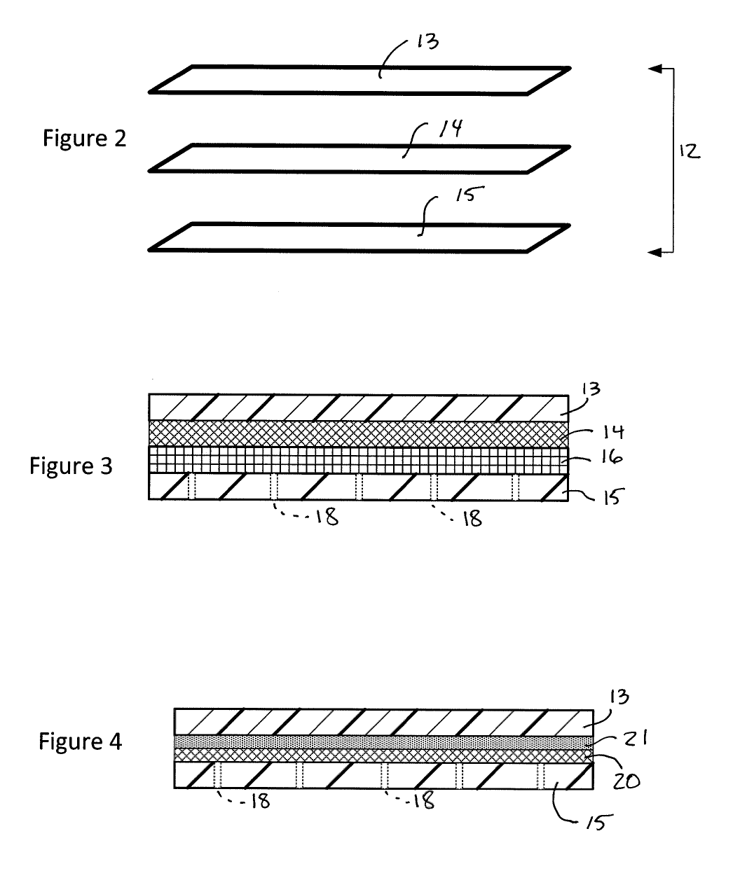

It exemplary embodiments, the water permeable wound side layer is a perforated polymer film which can be selected from the same group as the air side later. In FIGS. 3 and four, the perforations are illustrated (not to scale) by the perpendicular openings 18

In a particularly advantageous embodiment, the wound side layer is a cotton pad 16 (e.g. FIG. 3) bonded to a perforated film selected from the group consisted of biaxially oriented polyester (e.g., Mylar) and a fluorinated ethylene polymer (e.g., polytetrafluoroethylene, “PTFE,” Teflon®).

FIG. 3 is a cross⁻sectional view in somewhat more details than FIG. 2. The air side layer 13 is adjacent to the gel 14, and the perforated polymer film forms the wound side later 15, with a layer of cotton 16 between the gel and the perforated wound side layer

For wound treatment purposes, it can be advantageous for the gel 14 to include an electrolyte, which can be present in any desired soluble amount depending upon the intended purpose. Particular embodiments can include solutions that are common or standard in the medical context such as 0.9% sodium chloride (“saline”), or 0.45% sodium chloride concentration (“half saline”).

In other embodiments, the topical dressing 12 can incorporate medication in the gel layer 14. Appropriate medications can include anesthetics, analgesics, antiseptics, and anti-microbials (e.g. antibiotics, antifungal agents and antiviral agents).

Commonly topical analgesics include nonsteroidal anti-inflammatory drugs, lidocaine, capsaicin, amitriptyline, glyceryl trinitrate, opioids, menthol, pimecrolimus, and phenytoin.

Topical antiseptics are antimicrobial agents that kill, inhibit or reduce the number of microorganisms and are thought to be essential for wounds infection control. Commonly used products contain isopropyl or ethyl alcohol, povidone iodine, poloxamer iodine, benzalkonium chloride, benzethonium chloride, or chlorhexidine gluconate as a single agent or in combination with alcohol.

Common topical antibiotics include neomycin, polymyxin B, and bacitracin, which are often used as a “triple antibiotic” combination.

Common topical antifungals include clotrimazole, miconazole, ciclopirox, econazole, ketoconazole, and oxiconazole.

Common topical antivirals include acyclovir, docosanol, and penciclovir.

In another advantageous embodiment, the dressing includes a desiccated silicon oxide gel precursor rather than a hydrated gel. FIG. 4 illustrates this embodiment in cross⁻section. The dressing again includes the air side water impermeable layer 13, and the water permeable wound side layer 15, but instead of the hydrated gel, includes a desiccated gel 20 maintained in the dressing by an adhesive 21. The adhesive layer 21 is most often placed on the air side layer 13 to avoid interfering with the porous or perforated nature of the wound side layer 15. This is a convenience rather than a necessity, however, and appropriate adhesives (compositions and amounts) can hold the desiccated gel 20 against the layer 15 (wound side). A number of silicone-based adhesives are appropriate for medical use as are common hot melt adhesives (e.g., ethylene vinyl acetate polymers, “EVA”). Medical grade super glues (e.g., cyanoacrylates) are appropriate in some cases to form the edge seal 17.

The respective layers 13 and 15 can be formed up the same materials as in the first embodiment, and the desiccated silicon oxide gel precursor can again be fumed silica.

In many cases an appropriate medication that is likewise capable of being maintained in a dried or desiccated form can be included in or with the desiccated gel precursor.

The dressing 12 can include an appropriate skin-suitable adhesive on the exterior face (wound side face) of the wound side layer. Depending upon the adhesive, it can cover the entire wound side face of the dressing 12, or part of the wound side face, such as a pattern that reduces the overall amount of adhesive, but is sufficient to hold the dressing in place for its intended purpose and duration.

In some cases, the skin-suitable adhesive can be positioned on the perimeter of the wound side face (e.g., at 23 in FIG. 1) in a manner that tends to follow the sealed perimeter 17.

Typical skin-suitable adhesives for a dressing such as this include (those described to hold the desiccated gel in place, with the proviso that the most suitable adhesive will have minimal or no detrimental effect on the layers, the gel or any medications included in the gel. Other medical adhesives, which can be used for both the gel and the dressing, include acrylics, hydrocolloids, hydrogels, rubber-based adhesives, and polyurethane. Acrylics provide a secure anchor, but can cause skin tears on removal. Hydrocolloids are appropriate for skin contact, and can complement the gel aspects of the invention, but they can cause allergic reactions and reduce skin barrier function.

Hydrogels are suitable for “low trauma” bandaging, and are highly breathable and flexible, but their bonding weakens as they absorb fluid from a wound.

Rubber base adhesives have relatively low strength and are used mainly on surgical tapes and bandages, and can cause skin stripping to some extent when removed. Polyurethane is a suitable adhesive from a strength standpoint but is generally avoided in wound care because of the potential for stripping skin.

The advantages of the embodiment with the desiccated gel include lower weight and potentially longer shelf life depending upon circumstances. The desiccated-form dressing can also be somewhat thinner before the gel is hydrated, thus offering advantages for storage and transportation which in turn provide advantages in efficiency and cost.

In another aspect, the invention is a method of treating a wound that includes the steps of applying water to a desiccated gel precursor that is based upon a silicon oxide composition maintained in a dressing between an air side water impermeable layer and a water permeable wound side layer. Thereafter, the wound side layer of the dressing can be applied to the wound.

As noted elsewhere herein, the step of adding water can include the step of placing the desiccated dressing on a weeping or bleeding wound so that the water and related fluids hydrate the desiccated gel and produce the desired effect.

As in the other embodiments, fumed silica is an exemplary desiccated gel precursor to which the water is added. As in the other embodiments, the dressing can contain an appropriate medication that is otherwise consistent with the dressing and the hydrating step.

In yet another embodiment, the invention is a method of treating a wound by applying a dressing to the wound in which the dressing contains a hydrated gel that has been hydrated by adding water to a desiccated gel precursor that is based upon a silicon oxide composition. The desiccated gel precursor is maintained between an air side water impermeable layer and a water permeable wound side layer in the wound dressing.

Prior to the hydrating step, the desiccated gel can be applied to adhesive on the air side layer, and the gel precursor can be fumed silica.

As in the previous embodiments, the step of applying the dressing to the wound can include applying a dressing that includes a medication which as in the other embodiments can be selected from the group consisting of anesthetics, analgesics, antiseptics, and antimicrobials such as antibiotics, antifungal agents, and antiviral compositions.

The invention provides a number of advantages in comparison to conventional bandages. Because the gel can be supplied at concentrations as high as 99% water, a concentration can be selected that minimizes or eliminates fluid loss at a wound site. In particular, the environment can be designed to mimic blood chemistry in at least these aspects, and can also be provided at the same salt concentration as human blood, or any other needed concentration.

The same relationship applies to pH, which can be modified as desired.

The moist nature of the gel helps prevent dressings from adhering to wound surfaces because the dressing helps prevent plasma from drying and forming an adhesive surface. This in turn reduces demand on nurses or other caregivers because the device can remain in place longer than conventional dressings.

The device can be used for topical drug delivery of medication such as antibiotics (likewise reducing demands on caregivers) and can thus reduce the need for secondary delivery techniques in some circumstances (i.e., decreasing or avoiding topical, oral, or IV dosages).

In the same manner, the dressing helps reduce pain because of its basic avoidance of sticking to a wound, but the dressing can also be supplied with analgesics which can in turn reduce or eliminate the need for secondary pain medication that when applied in other techniques (e.g. orally or by IV) may otherwise affect the whole body unnecessarily.

All of these advantages make the device cost-effective because of it's reduced demand on staff and avoidance of other medications.

In an urgency aspect (first responders, military battlefield use, etc.), the dehydrated form of the dressing provides a smaller, lighter package that can be rehydrated at the point of use.

In some cases, the dehydrated or desiccated form can be applied to a bleeding or weeping wound so that the body fluid can provide the hydration for the gel, as well as the other advantages of the dressing.

In the drawings and specification there has been set forth a preferred embodiment of the invention, and although specific terms have been employed, they are used in a generic and descriptive sense only and not for purposes of limitation, the scope of the invention being defined in the claims.

1. A topical dressing for wound recovery comprising:
a water impermeable air side layer,
a water permeable wound-side layer; and
a gel based upon a silicon oxide composition positioned between said air side layer and said wound side layer.

2. A topical dressing according to claim 1 wherein said silicon oxide composition comprises fumed amorphous silicon dioxide with a particle size of between about 5 and 50 nanometers.

3. A topical dressing according to claim 1 wherein said air side layer is selected from the group consisting of polymers, metal foils, combinations of polymers, and combinations of polymers and metal foils.

4. A topical dressing according to claim 3 wherein said air side layer is a polymer selected from the group consisting of polyester, PTFE, polyethylene, polypropylene, polyamide, EVOH, and combinations thereof.

5. A topical dressing according to claim 1 wherein said water permeable wound side layer is a perforated polymer film selected from the group consisting of polyester, PTFE, polyethylene, polypropylene, polyamide, EVOH, and combinations thereof.

6. A topical dressing according to claim 1 wherein said water permeable wound side layer is a cotton pad bonded to a perforated film selected from the group consisting of biaxially oriented polyester film and polyfluorinated ethylene.

7. A topical dressing according to claim 1 wherein said gel includes an electrolyte.

8. A topical dressing according to claim 7 wherein said electrolyte is present in said gel in any soluble amount.

9. A topical dressing according to claim 8 wherein the electrolyte is 0.9% sodium chloride.

10. A topical dressing according to claim 8 wherein said electrolyte is 0.45% sodium chloride.

11. A topical dressing according to claim 1 wherein said gel is buffered.

12. A topical dressing according to claim 11 wherein said gel is buffered to a pH of between about 3 and 12.

13. A topical dressing according to claim 12 wherein said gel is buffered with a composition selected from the group consisting of sodium bicarbonate, calcium carbonate, magnesium oxide, monosodium phosphate, bisodium phosphate, and mixtures thereof.

14. A topical dressing according to claim 1 wherein said gel includes a medication selected from the group consisting of anesthetics, analgesics, antiseptics, antifungals and antimicrobials.

15. A topical dressing according to claim 1 wherein said air side layer and said wound side layer are sealed to one another with said gel in between.

16. A topical dressing for wound recovery comprising:
an air side water impermeable layer;
a water permeable wound side layer;
an adhesive layer on the wound side face of said air side layer; and
a desiccated silicon oxide gel precursor on said adhesive.

17. A topical dressing according to claim 16 wherein said air side layer is selected from the group consisting of polymers, metal foils, combinations of polymers, and combinations of polymers and metal foils.

18. A topical dressing according to claim 17 wherein said air side layer is a polymer selected from the group consisting of polyester, PTFE, polyethylene, polypropylene, polyamide, EVOH, and combinations thereof.

19. A topical dressing according to claim 16 wherein said water permeable wound side layer is a perforated polymer film selected from the group consisting of polyester, PTFE, polyethylene, polypropylene, polyamide, EVOH, and combinations thereof.

20. A topical dressing according to claim 16 wherein said water permeable wound side layer is a cotton pad bonded to a perforated film selected from the group consisting of biaxially oriented polyester film and polytetrafluoroethylene.

21. A topical dressing according to claim 16 wherein said gel precursor includes a medication selected from the group consisting of anesthetics, analgesics, antiseptics, antifungals and antimicrobials.

22. A topical dressing according to claim 16 wherein said air side layer and said wound side layer are sealed to one another with said gel precursor therebetween.

23. A topical dressing according to claim 16 wherein said gel precursor comprises fumed silica.

24. A method of treating a wound comprising:
adding water to a desiccated silicon oxide-based gel precursor based upon a silicon oxide composition maintained in a dressing between an air side water impermeable layer and a water permeable wound side layer; and
applying the wound side layer of the dressing to a wound.

25. A method of treating a wound according to claim 24 comprising adding water to fumed silica.

26. A method of treating a wound according to claim 25 comprising adding water to fumed silica that includes a medication selected from the group consisting of anesthetics, analgesics, antiseptics, antifungals and antimicrobials.

27. A method of treating a wound comprising applying a dressing to a wound, and in which the dressing contains a hydrated gel that has been hydrated by adding water to a desiccated gel precursor based upon a silicon oxide composition in which the desiccated gel precursor is maintained between an air side water impermeable layer and a water permeable wound side layer in the wound dressing.

28. A method of treating a wound according to claim 27 comprising applying a dressing in which the desiccated gel is applied to an adhesive on the air side layer.

29. A method of treating a wound according to claim 27 comprising applying a dressing to a wound in which the gel precursor is fumed silica.

30. A method of treating a wound according to claim 27 comprising applying a dressing to a wound in which the fumed silica includes a medication selected from the group consisting of anesthetics, analgesics, antiseptics, antifungals and antimicrobials.
